(19) United States
(12) Patent Application Publication
(10) Pub. No.: US 2003/0118557 A1
(43) Pub. Date: Jun. 26, 2003

(54) Axon repair

(76) Inventors: Howard Bomze, Durham, NC (US); Ketan Bulsara, Durham, NC (US); Bermans Iskandar, Madison, WI (US); Pico Caroni, Muttenz (CH); Pate Skene, Chapel Hill, NC (US)

Correspondence Address: NIXON & VANDERHYE, PC, 1100 N GLEBE ROAD, 8TH FLOOR, ARLINGTON, VA, 22201-4714, US

(21) Appl. No.: 10/268,967

(22) Filed: Oct. 11, 2002

Related U.S. Application Data
(60) Provisional application No. 60/328,102, filed on Oct. 11, 2001.

(51) Int. Cl. A61K 48/00; A61K 39/395; A61K 9/127; C12N 15/86

(52) U.S. Cl. 424/93.2; 514/12; 424/450; 514/44; 424/178.1; 435/456

(57) ABSTRACT

The present invention relates generally to methods of effecting axon repair.

The present application claims priority from U.S. Provisional Application No. 60/328,102, filed Oct. 11, 2001, the contents of which are incorporated herein by reference.

This invention was made with Government support under EY11475 awarded by the National Institutes of Health. The Government has certain rights in the invention.

### TECHNICAL FIELD

The present invention relates generally to methods of effecting axon repair.

### BACKGROUND

Neurons form functional connections within the nervous system by extending long fibers, call exons, to establish synaptic contacts with other cells. Axons damaged in the mammalian brain and spinal cord do not ordinarily regenerate. As a result, CNS trauma, stroke or degenerative disease leads to permanent blindness, paralysis or other loss of function. Research over the last 20 years has identified two major hurdles to CNS regeneration. One is the presence on CNS glial cells of proteins and proteoglycans that can directly inhibit axon extension (Fidler et al, J. Neurosci. 19:8778-8788 (1999), Goldberg et al, Nature 403:369-370 (2000), Chen et al, Nature 403:434-439 (2000), Davies et al, J. Neurosci. 19:5810-5822 (1999)). Even axons that initiate effective regeneration may be stopped when they encounter these inhibitory cues. The other major impediment to CNS repair is that axotomized neurons often fail to activate a program of gene expression adequate to support regeneration. In particular, genes coding for protein components of axonal growth cones—the motile tips of extending axons—are generally suppressed in mature neurons, but are readily reactivated by peripheral nerve injury (Skene et al, J. Cell Biol. 89:96-103 (1981), Skene, Ann. Rev. Neurosci. 12:127-156 (1989), Caroni, Bioessays 19:767-775 (1997)). Following CNS injury, however, at least some of these growth-associated proteins (GAPs) remain suppressed in the majority of injured neurons (Skene et al, J. Cell Biol. 89:96-103 (1981), Kalil et al, J. Neurosci. 6:2563-2570 (1986), Doster et al, Neuron 61-13 (1991), Schreyer et al, J. Neurobiol. 24:959-970 (1993), Fernandes et al, J. Comp. Neurol. 414:495-510 (1999)).

The significance of this differential gene regulation has been tested by grafting segments of peripheral nerve into the brain or spinal cord, providing CNS axons with a supportive environment for axon growth (David et al, Science 214:931-933 (1981), Richardson et al, J. Neurocytol. 13:165-182 (1984), So et al, Brain Res. 328:349-354 (1985), Friedman et al, J. Neurosci. 5:1616-1625 (1985)). Although some CNS axons regrow for long distances into these nerve grafts, regeneration occurs only from neurons whose cell bodies are located within a few millimeters of the lesion site. Such proximal lesions can activate GAP expression in a subset of the injured neurons, and regenerating axons arise exclusively from these GAP-expressing cells (Campbell et al, Exp. Brain Res. 87:67-74 (1991), Schaden et al, J. Neurobiol. 25:1570-1578 (1994).

The central role of axotomy-induced genes in regeneration has been demonstrated most elegantly for dorsal root ganglion (DRG) neurons, which are unique in having both a long CNS axon that ascends the spinal cord and a second axon branch that projects through a peripheral nerve. Interruption of DRG spinal axons fails to induce GAP genes (Schreyer et al, J. Neurobiol. 24:959-970 (1993), Chong et al, J. Neurosci. 14:4375-4384 (1994)), and the injured axons are unable to regenerate (Richardson et al, Nature 309:791-793 (1984)). However, when the spinal cord lesion is combined with peripheral nerve injury, the neurons become competent to regenerate their spinal axons into a nerve graft (Richardson et al, Nature 309:791-793 (1984)). In fact, recent studies show that these DRG axons can regenerate for a substantial distance within the native environment of the spinal cord (Davies et al, J. Neurosci. 19:5810-5822 (1999), Neumann et al, Neuron 23:83-91 (1999)).

These observations show that genes activated by peripheral nerve injury can be crucial in determining the success or failure of CNS axon regeneration. A fundamental question is which of these gene(s) are responsible for triggering regeneration. The most extensively studied example has been the gene for GAP-43, an abundant component of axonal growth cones widely correlated with successful axon regeneration (Skene, Ann. Rev. Neurosci. 12:127-156 (1989), Caroni, Bioessays 19:767-775 (1997)). Loss of GAP-43 impairs axon extension in response to cell adhesion molecules (Meiri et al, J. Neurosci. 18:10429-10437 (1998)), increases susceptibility to growth cone collapse by CNS myelin (Aigner et al, J. Cell Biol. 128:647-660 (1995)), and disrupts axon guidance and synaptic organization during development (Strittmatter et al, Cell 80:445-452 (1995), Maier et al, Proc. Natl. Acad. Sci. USA 96:9397-9402 (1999), Zhu et al, Exp. Neurol. 155:228-242 (1999)). In adult neurons, overexpression of GAP-43 enhances sprouting at axon terminals (Caroni, Bioessays 19:767-775 (1997), Caroni et al, J. Cell Biol. 136:679-692 (1997), Buffo et al J. Neurosci. 17:8778-8791 (1997)). Replacing GAP-43 alone, however, is not sufficient to trigger regeneration (Neumann et al, Neuron 23:83-91 (1999), Buffo et al J. Neurosci. 17:8778-8791 (1997)).

The present invention results, at least in part, from the use of an in vitro assay to search for additional genes that can mimic the effects of peripheral nerve injury in stimulating axon elongation by DRG neurons. Genes revealed by this search are sufficient to induce regeneration of spinal cord axons in vivo.

### SUMMARY OF THE INVENTION

The present invention relates generally to methods of effecting axon repair. More specifically, the invention relates to a method of effecting axon repair that involves use of GAP-43 in combination with other growth-associated genes to promote CNS axon regeneration.

Objects and advantages of the present invention will be clear from the description that follows.

### BRIEF DESCRIPTION OF THE DRAWINGS

FIG. 1. GAP-43 and CAP-23 increase the propensity of adult neurons for axon growth in vitro. DRG neurons were isolated from control (non-transgenic) adult mice or from transgenic mice expressing high levels of GAP-43 and/or CAP-23 in adult neurons. For comparison, neurons were isolated from non-transgenic animals that had undergone a peripheral nerve lesion 4 days before removal of the ganglia. The graph indicates the percentage of adult neurons that extended axonal processes by 24 hours after plating.

FIGS. 2A-2E. Combined expression of GAP-43 and CAP-23 triggers an elongating mode of axon extension. DRG neurons from non-transgenic, wild-type mice (wt), or from animals expressing the indicated transgenes, were isolated and plated as in FIG. 1. Ganglia were isolated with no prior manipulation (FIGS. 2A-2D), or 4 days following a crush injury to the sciatic nerve (periph. lesion, FIG. 2E). The photographs at left show phase-contrast images of representative neurons from each type of culture. Axons from the GAP-43/CAP-23 transgenic animals, and from neurons that have responded to a peripheral nerve lesion, extend beyond the borders of these images. The cells depicted were stained with antibodies against tubulin (wild-type animals) or the relevant transgene products. For the doubly transgenic cell, staining is for GAP-43. The scale bar in each image represents 100 μm. Histograms at right depict the length of the longest process for individual neurons in each culture. Naive ganglia from non-transgenic control animals extend primarily short (100-200 μm) axons, while peripheral nerve injury elicits the extension of very long (>300 μm) axons. Expression of CAP-23 alone fails to trigger the extension of long axons comparable to those induced by peripheral nerve injury. While GAP-43 leads to the emergence of a small population neurons with very long axons (>300 μm), the majority of neurons continue to extend the shorter axons (100-150 μm) characteristic of naive adult neurons. In contrast to either protein alone, simultaneous expression of GAP-43 and CAP-23 triggers the extension of very long axons by the majority of DRG neurons, which mimics the effect of peripheral nerve injury.

FIG. 3. Stepwise induction of axon elongation by GAP-43 and CAP-23. DRG neurons were analyzed for axon growth in vitro as for FIG. 2. The number of branch points and total axon length were measured for the longest process for individual neurons; the graph shows the mean branch number and length ±95% confidence interval for each condition. For non-transgenic animals (open symbols) naive neurons (open circle) extend relatively short, highly branched axons, while peripheral nerve injury (open square) induces the extension of very long, sparsely branched axons. For naive neurons isolated from transgenic animals with no prior nerve injury (closed symbols), expression of either GAP-43 (closed square) or CAP-23 (closed circle) reduces axonal branching, but does not trigger axon elongation. In contrast, combined expression of these two growth-associated proteins (closed diamond) mimics the effects of peripheral nerve injury in triggering the elongating mode of growth.

FIGS. 4A and 4B. Expression of GAP-43 and CAP-23 and regeneration of spinal axons by large mechanosensory DRG neurons of transgenic mice in vivo. FIG. 4A. Immunofluorescent staining shows the presence of chicken CAP-23 (blue) or GAP-43 (green) in dorsal column axons in longitudinal sections of spinal cord from adult transgenic mice. The left panel was taken at the border between the dorsal columns (left side of the image) and the gray matter of the dorsal horn. Note that CAP-23 is present in dorsal columns axons, and also in neurons of the dorsal horn. The right panel illustrates GAP-43 positive axons in the dorsal columns. The lower panels illustrate control sections stained with no primary antibody. FIG. 4B. Neuron cell bodies in the dorsal root ganglion (DRG) of an animal transgenic for both GAP-43 (green) and CAP-23 (blue). Both proteins are expressed in many large DRG neurons. Three of these cells also contain the retrograde axonal tracer diI (red), indicating that they have regenerated their spinal axons through a peripheral nerve segment placed in the dorsal columns 5 weeks earlier. All three cells displayed strong cell body staining for both GAP-43 and CAP-23. The enlarged views at right illustrate the separate images of GAP-43 and CAP-23 immunofluorescence for one of these neurons.

FIGS. 5A and 5B. Replacement of GAP-43 and CAP-23 permits regeneration of spinal sensory axons in vivo. FIG. 5A. Schematic of the experiment. Axons ascending in the dorsal columns of the spinal cord were interrupted in adult non-transgenic (wild-type) mice or mice expressing both the GAP-43 and CAP-23 transgenes (transgenic). A segment of peripheral nerve was removed from the left sciatic nerve, severing the peripheral axons of DRG neurons on one side. The nerve segment was then grafted into the spinal cord lesion site, spanning the dorsal columns on both sides of the midline. One to four months later, a fluorescent tracer (diI, depicted in red) was applied to the distal end of the graft. Axons that have regenerated at least 5 mm into the nerve graft are able to take up the fluorescent tracer and transport it retrogradely to the neuron cell bodies. FIG. 5B summarizes the mean number of labeled neurons detected in the lumbar dorsal root ganglia. DRG neurons subjected to peripheral nerve injury at the same time as the dorsal column lesion (Periph. lesion, open bars) are able to regenerate their spinal axons in to the nerve grafts. In non-transgenic (wt) mice, neurons that have not responded to a peripheral nerve lesion (No Periph. lesion) fail to regenerate their spinal axons. Expression of GAP-43 and CAP-23 induces a 60-fold increase in the number of DRG neurons that can regenerate their spinal axons from the dorsal column lesion.

### DETAILED DESCRIPTION OF THE INVENTION

The present invention relates to a method of stimulating axon repair or regeneration comprising introducing into neuron cell bodies DNA sequence(s) that encode two or more members of a family of growth cone proteins that are typically missing or deficient in adult neurons. One key to the present invention is the use of a combination of sequences coding for two or more proteins with related, but complementary, functions in axonal growth cones. A second key feature of the present method is that it employs direct expression of the sequences of interest in the cell bodies of neurons the axons of which are to be stimulated to grow. Previous designs have sought to express genes for cytokines, neurotrophic factors, or other extracellular signalling molecules in glial cells or other non-neuronal cells. Those designs rely on the principle of expressing a secreted factor that may act secondarily on neurons to stimulate growth.

In a preferred embodiment, the DNA sequence(s) encode the proteins GAP-43 (also known as neuromodulin or B50) and CAP-23 (also known as NAP22 or BASP1). These proteins have related functions, in that each protein modulates the localization and activities of phosphoinositide lipid signaling molecules, calmodulin, and actin in axonal growth cones. They are complementary because the protein domains responsible for membrane targeting, and for interactions with lipid and protein signaling molecules, differ between GAP-43 and CAP-23. Other proteins that share these properties include MARCKS, MacMARCKS, and paralemmin. Such sequences can be used instead of, or in addition to, GAP-43 and CAP-23.

Exogenous DNA constructs that direct expression of selected genes can employ any viral, plasmid, or other vector capable of directing gene expression in neurons. In one specific embodiment, DNA sequences coding for GAP-43 (or analog thereof—see, for example, U.S. Pat. No. 6,106,824) and for CAP-23 (or analogs thereof) are inserted into recombinant viruses that are taken up by injured axons and transported retrogradely to the corresponding neurons cell bodies. Such viruses include, but are not limited to, known neurotropic virus families, such as herpes, sindbis, polio, pseudorabies, and adenoviruses. Similar results can be obtained with any other vehicles that can be used to deliver encoding sequences into target neurons.

Axon repair using the combination of growth associated proteins can be effected, for example, using direct gene therapy. Essentially any viral or non-viral vector can be used to introduce the appropriate combination of genes (or the proteins themselves) into injured or damaged neurons. As indicated above, the encoding sequences can be introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. Use of defective viral vectors allows for administration to cells in a specific, localized area, without concern that the vector can infect other cells.

Alternatively, the vector can be introduced in vivo by lipofection. Synthetic cationic lipids designed to limit the difficulties and dangers encountered with liposome mediated transfection can be used to prepare liposomes for in vivo transfection of the present sequences (Felgner, et. al., 1987, Proc. Natl. Acad. Sci. U.S.A. 84:7413-7417; see Mackey, et al., 1988, Proc. Natl. Acad. Sci. U.S.A. 85:8027-8031)). The use of cationic lipids can promote encapsulation of negatively charged nucleic acids, and also promote fusion with negatively charged cell membranes (Felgner and Ringold, 1989, Science 337:387-388). Lipofection into the nervous system in vivo has been achieved (Holt, Neuron 4:203-214 (1990)). The use of lipofection to introduce exogenous genes into the nervous system in vivo has certain practical advantages. Molecular targeting of liposomes to specific cells represents one area of benefit. Directing transfection to limited neuronal types is particularly advantageous in a tissue with such cellular heterogeneity as the brain. Lipids can be chemically coupled to other molecules for the purpose of targeting. Targeted peptides, e.g., hormones or neurotransmitters, and proteins such as antibodies, or non-peptide molecules can be coupled to liposomes chemically.

The encoding sequences can also be introduced as a naked DNA plasmid. This is particularly the case where an axon has been cut, thus exposing the axonal cytoplasm. Any DNA in proximity to the cut axon may be taken up and transported via the axon transport mechanism to the cell body, where the plasmid can enter the nucleus.

Encoding sequences of the invention can also be introduced via a DNA vector transporter (see, e.g., Wu et al, J. Biol. Chem. 267:963-967 (1992); Wu and Wu, J. Biol. Chem. 263:14621-14624 (1988); Hartmut et al., Canadian Patent Application No. 2,012,311, filed Mar. 15, 1990).

According to the present invention, the encoding sequence can be present in the vector under the control of any promoter. Preferably, the promoter provides for high level expression of the encoding sequence for a finite period of time. Thus, the preferred promoters are promoters that are active for a short time, such as viral promoters for early genes. In a specific embodiment, the human cytomegalovirus (CMV) immediate early promoter can be used to effect transient expression. Alternatively, an inducible promoter can be used. Promoters that can be used include, but are not limited to, the SV40 early promoter region (Benoist and Chambon, Nature 290:304-310 (1981)), the promoter contained in the 3′ long terminal repeat of Rous sarcoma virus (Yamamoto et al, Cell 22:787-797 (1980)), the herpes thymidine kinase promoter (Wagner et al, Proc. Natl. Acad. Sci. U.S.A. 78:1441-1445 (1981)), the regulatory sequences of the metallothionein gene (Brinster et al, Nature 296:39-42 (1992)); and the transcriptional control regions that exhibit tissue specificity and that have been utilized in transgenic animals.

Axon repair in accordance with the invention can also be effected by targeting stimulation of, for example, GAP-43 and CAP-23 expression using pharmaceuticals that activate the endogenous genes (e.g., GAP-43 and CAP-23).

Axon repair can also be effected using mimics, e.g., GAP-43 and CAP-23 mimics. Suitable mimics include peptides or fusion proteins designed to mimic the biochemical actions of GAP-43 and CAP-23, or other MARCKS-related proteins.

In a further embodiment, the present invention relates to a method of screening for drugs or other treatments that can activate GAP-43, CAP-23 or related genes. By demonstrating that it is a combination of genes that leads to axon regeneration, basis is provided for an assay to detect agents for use in promoting axon regeneration. Drugs or other treatments can be tested, for example, by application to adult neurons in vitro or in vivo, and monitoring for the ability to elicit co-expression of, for example, GAP-43 and CAP-23. Measurements of expression can employ any standard procedures for measuring gene expression (Northern blotting, RT-PCR, in situ hybridization, DNA arrays, etc.).

In yet another embodiment, the invention relates to an in vitro assay for rapid evaluation of neuronal ability to support regeneration. Provided herein is an in vitro assay that accurately predicts the ability of adult neurons to support effective axon regeneration in vivo. In accordance with this assay, the percentage or cells that extend processes >2 cell body diameters is measured, length of the longest axonal process is measured, as is the number of branch points formed from the longest process. (See also Smith and Skene, J. Neuro. 17:646 (1997).)

The present method is applicable to many situations in which axon regrowth can facilitate functional recovery: spinal cord injuries, head trauma, stroke, degenerative diseases, among other insults that interrupt CNS axons. In addition, this is applicable to lesions that affect the centrally projecting axons of DRG neurons within the dorsal roots (e.g., dorsal root avulsions, “pinched” roots, etc.). Expression of GAP-43 and CAP-23, or small combinations of other growth-associated proteins, stimulates effective repair of dorsal root lesions.

The present invention provides methods for the treatment of nerve damage associated with a lesion or a disease or dysfunction of the nervous system. Preferably, the subject treated is a human, however, the methods of the invention are also applicable to non-human mammals.

### EXAMPLE

Experimental Details

Transgenic mouse lines expressing chicken GAP-43 or CAP-23 under the control of a neuron-specific Thy-1 promoter were derived from line wt3 (GAP-43) and line c11 (CAP-23), previously described (Caroni et al, J. Cell Biol. 136:679-692 (1997), Aigner et al, Cell 83:269-278 (1995)). Previous studies showed that the avian proteins are effective in modulating phosphoinositide distribution and actin dynamics, and can stimulate axonal sprouting in mammalian neurons (Frey et al, J. Cell Biol. 149:1443-1453 (2000), Laux et al, J. Cell Biol. 149:1455-1471 (2000)). The transgenic lines were chosen so that the levels of transgene expression in adults is similar to the expression of endogenous GAP-43 and CAP-23 in developing neurons. Transgene expression begins at approximately postnatal day 6 and continues through adult life. Mice were genotyped using standard PCR methods. The primers (5′-CCAACAGCGGAGAAAAAAGGG-3′) and (5′-TCTTCTTTCACCTCTTCCTGC-3′) amplify a 380 bp DNA fragment from the chicken GAP-43 transgene; for the CAP-23 transgene, the primers (5′-AAGGATGCTCAGGTCTCTGC-3′) and (5′-GTCTTTTTGGCTTCCCCTTCC-3′) amplify a 317 bp fragment. Neither set of primers amplifies the corresponding endogenous gene from mouse. Mice positive for each transgene were mated to ensure heterozygosity in the experimental animals and to generate doubly transgenic animals. Control animals were generated as littermates in the same breedings.

For the in vitro analysis, dorsal root ganglion (DRG) neurons from adult mice (>8 weeks) were dissociated essentially as described (Smith et al, J. Neurosci. 17:646-658 (1997)), and centrifuged at 200×g for 10 minutes through a cushion of 10% Ficoll in F14 culture medium to remove myelinated axons, cellular debris and non-neuronal cells. Neurons were resuspended in serum-free F14 medium containing N1 supplements, and plated on polylysine/laminin coated glass coverslips as described (Smith et al, J. Neurosci. 17:646-658 (1997)). Cells from 12-14 ganglia were plated in 12 wells of a standard 24-well plate. After 18-24 hours, cultures were fixed in 4% paraformaldehyde for 30 minutes at room temperature, washed and stained with antibodies to detect neurons expressing chicken CAP-23 (monoclonal antibody 15C1) and chicken GAP-43 (rabbit polyclonal antibody) (Caroni et al, J. Cell Biol. 136:679-692 (1997), Aigner et al, Cell 83:269-278 (1995)). To visualize all neuronal processes, cultures were stained with antibodies to β III tubulin (MAB 1637; Chemicon, Temecula, Calif.). Cells were viewed with a CCD camera and analyzed with IPLab 3.2 for the Macintosh (Scanalytics, Inc., Fairfax, Va.). Only neurons that stained strongly for the appropriate transgene(s) were analyzed. In control cultures, cells that stained strongly for the neuron-specific β III tubulin were analyzed. Cells with processes greater than 2 cell body diameters were scored. The length of the longest process for each cell, and the number of branches formed along that process, were then measured.

For analysis of spinal axon regeneration in vivo, DRG axons were transected in the dorsal columns on both sides of the spinal cord in adult mice, at the level of the cervico-thoracic junction (>4 weeks). A segment of sciatic nerve on one side was resected and grafted into the spinal cord lesion site (Richardson et al, Nature 309:791 (1984), Richardson et al, J. Neurocytol. 15;585 (1986)). After 1-4 months, the fluorescent tracer diI was introduced into the nerve graft 5 mm from the spinal cord. After another 5 days, the animals were perfused transcardially with 4% paraformaldehyde, and the dorsal root ganglia removed and post-fixed in 30% sucrose. Thirty micron cryostat sections were evaluated under fluorescent microscopy. Fluorescently labeled cells were counted, and differences due to genotype and peripheral nerve injury were analyzed by two-way ANOVA followed by Fisher's protected least significant difference posthoc test (StatView; SAS Inc., Cary, N.C.). To identify cells expressing the transgenes, cryostat sections were stained with antibodies against chicken CAP-23 and GAP-43, followed by secondary antibodies labeled with Alex Fluor 488 and Alex Fluor 350 (Molecular Probes, Eugene, Oreg.). Sections were viewed with narrow-band filter sets for each of the labels; control sections stained with no primary antibodies, or with only one primary antibody, confirmed that there was no detectable cross-over of signals.

Results

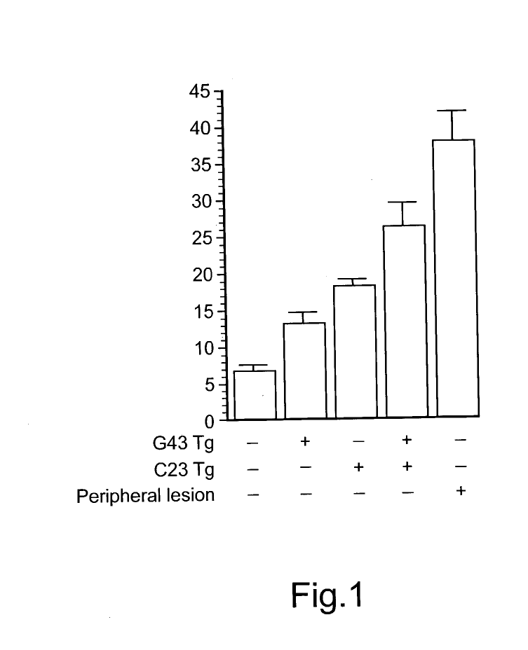

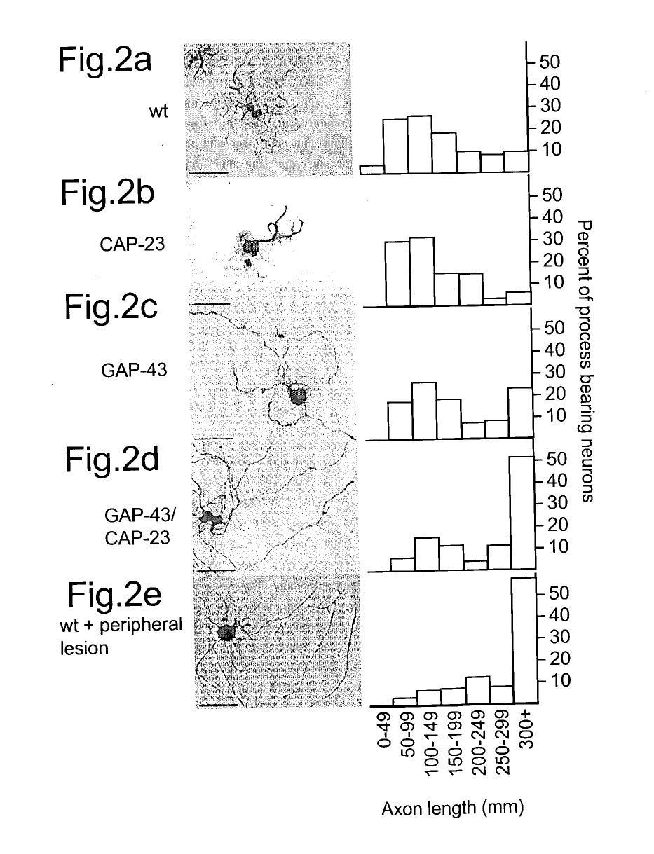

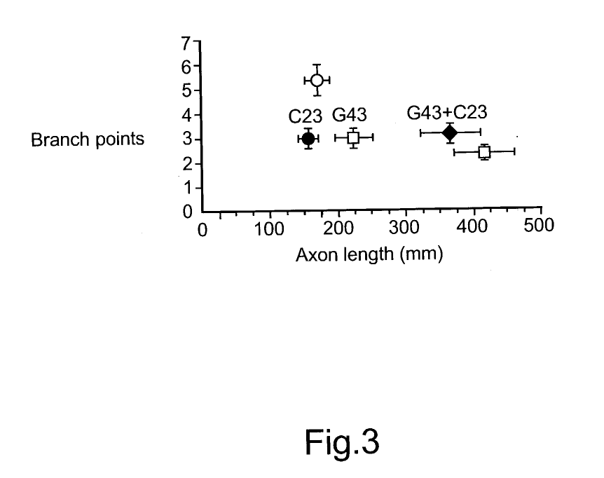

To identify genes responsible for the onset of axon regeneration, a short-term in vitro assay was used that monitors a transcription-dependent switch in axon extension induced in DRG neurons by axon injury (Smith et al, J. Neurosci. 17:646-658 (1997)). Neurons are removed from adult animals and cultured for 18-24 hours (Smith et al, J. Neurosci. 17:646-658 (1997)). Because the neurons are axotomized during this removal, they will eventually respond by inducing the full complement of growth-associated genes (Smith et al, J. Neurosci. 17:646-658 (1997)). Over the first 24 hours in culture, however, axon outgrowth depends only on genes that were already expressed in the neurons at the time of their removal from the animal (Smith et al, J. Neurosci. 17:646-658 (1997)). Neurons isolated from adult mice with no prior manipulation (naive neurons) supported a limited amount of outgrowth (Smith et al, J. Neurosci. 17:646-658 (1997)) and FIG. 1), characterized by the emergence of relatively short and highly branched axons (FIGS. 2 and 3). In contrast, neurons that had responded to a peripheral nerve lesion several days before removal were much more likely to extend axons (FIG. 1), and those axons were long and sparsely branched (FIGS. 2 and 3). This “elongating” growth resembles the extension required for nerve regeneration in vivo, and reflects the expression of genes induced by peripheral nerve injury (Smith et al, J. Neurosci. 17:646-658 (1997)).

To identify genes that trigger this regenerative growth, neurons were isolated from transgenic animals in which expression of specific growth-associated proteins is maintained in adult neurons (Caroni et al, J. Cell Biol. 136:679-692 (1997), Aigner et al, Cell 83:269-278 (1995)). Persistent expression of GAP-43 in adult DRG neurons increased the propensity of naive adult neurons to extend axons in the acute outgrowth assay (FIG. 1), but the majority of those axons remained relatively short, with a modal length of 100-150 μm (FIG. 2). Only a small fraction of the GAP-43-expressing cells extended long (>300 μm) axons of the sort induced by peripheral nerve injury (FIG. 2). To ensure that this was not due to limited expression of the transgene, the cultures were stained with an antibody against chick GAP-43. At least 80% of the DRG neurons stained intensely for transgene expression, and only those cells were included in the analyses reported here. The minimal effect of GAP-43 on the extension of long axons is consistent with earlier reports that GAP-43 alone is not sufficient to trigger regeneration of CNS axons in vivo (Neumann et al, Neuron 23:83-91 (1999), Buffo et al J. Neurosci. 17:8778-8791 (1997)). This implies that additional genes are involved in the transition from local axon arborization to elongating growth.

GAP-43 shares a number of features with another prominent growth cone component induced by peripheral nerve injury, CAP-23 (Wiederkehr et al, Experimental Cell Research 236:103-116 (1997)). Both GAP-43 and CAP-23 are members of a MARCKS-related group of acylated membrane proteins that interact with calmodulin, actin filaments, protein kinase C, and phosphoinositides (Wiederkehr et al, Experimental Cell Research 236:103-116 (1997), Mosevitsky et al, Biochimie 79:373-384 (1997), Maekawa et al, J. Biol. Chem. 274:21369-21374 (1999)). In transgenic mice, both GAP-43 and CAP-23 enhance local sprouting at axon terminals in vivo Caroni, Bioessays 19:767-775 (1997), Aigner et al, Cell 83:269-278 (1995)). A determination was therefore made as to whether CAP-23, alone or in combination with GAP-43, can contribute to the induction of axon elongation following peripheral nerve injury. As with GAP-43, persistent expression of CAP-23 increased the number of adult DRG neurons that extended axons in short-term cultures (FIG. 1), but did not elicit extension of long axons (FIG. 2). Combined expression of GAP-43 and CAP-23, however, induced a large population of DRG neurons to extend long (>300 μm) axons (FIG. 2).

The effects of co-expressing GAP-43 and CAP-23 were qualitatively different from the effects of either protein alone. While each protein alone acted primarily to reduce axon branching, simultaneous expression of these growth cone components triggered a dramatic increase in axon length (FIG. 3). Averaged over the entire population of DRG neurons, the effects of GAP-43/CAP-23 co-expression approximated the effects of peripheral nerve injury (FIG. 3), although there were small differences. Axons from the transgenic mice tended to be slightly shorter and branched somewhat more frequently than after peripheral nerve injury. The difference in axon length arose from the persistence of a small population of neurons with short (100-150 μm) axons in ganglia from the transgenic animals (FIG. 2). When this subpopulation was removed from the analysis, the average axon length for the remaining neurons from GAP-43/CAP-23 expressing animals (538±54 μm) was essentially identical to that for ganglia subjected to peripheral nerve injury (546±49 μm). However, the small difference in branching frequency persisted. Thus, co-expression of GAP-43 and CAP-23 triggered a transition in axon growth that is very similar—but not quite identical—to that evoked by the full complement of genes induced by peripheral nerve injury.

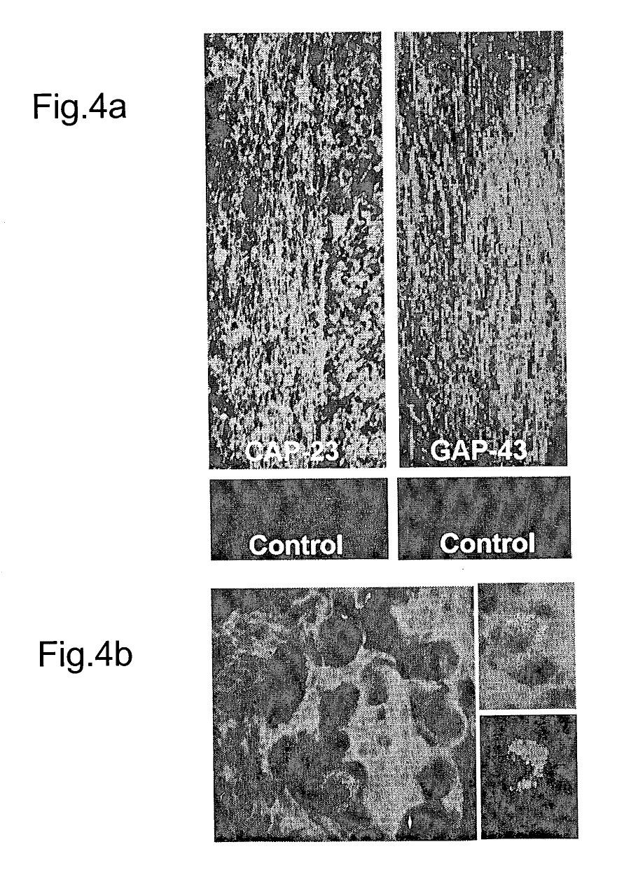

In vivo, one of the most striking consequences of peripheral nerve injury is that it enables DRG neurons to support regeneration of their axons in the spinal cord (Richardson et al, J. Neurocytol. 13:165-182 (1984), Neumann et al, Neuron 23:83-91 (1999)). These dorsal column axons arise from a specific population of large, mechanosensory neurons in the DRG. Immunostaining confirmed that the largest DRG neurons in our dissociated cultures 40 μm diameter) expressed the GAP-43 and CAP-23 transgenes at a frequency similar to other DRG neurons. Re-analysis of axon outgrowth for this subpopulation showed, furthermore, that the frequency of axon extension, and the mean axon length and number of axon branches for these neurons fall within the 95% confidence interval for the overall population of DRG neurons. This suggests that the elongating mode of axon growth can be elicited by GAP-43 and CAP-23 expression in the large mechanosensory cells, as well as in other classes of DRG neurons. Moreover, immunostaining of cryostat sections showed that the large DRG neurons expressed the GAP-43 and CAP-23 transgenes in vivo, and transported the proteins into their axons in the dorsal columns (FIG. 4). If expression of these proteins were sufficient to mimic the effects of peripheral nerve injury in stimulating regeneration in vivo, as it does in the in vitro assay, then DRG neurons in the transgenic animals should support significant regeneration of spinal axons in the absence of a peripheral nerve injury.

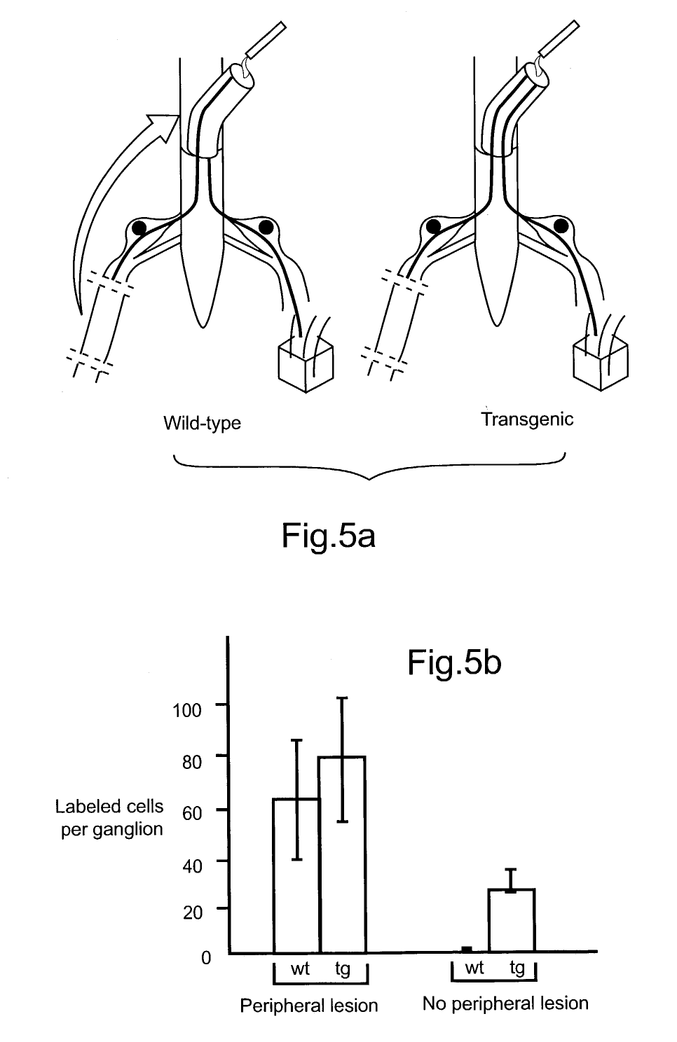

To test this possibility, spinal cord lesions that sever the central axons of DRG neurons were made in wild-type mice and in transgenic animals expressing both GAP-43 and CAP-23. Dorsal column axons were transected on both sides of the spinal cord, at the level of the cervico-thoracic junction. To provide the injured axons with an optimal environment for regrowth, a segment of peripheral nerve (sciatic) was resected on one side and the nerve segment was grafted into the spinal cord lesion site (Richardson et al, J. Neurocytol. 13:165-182 (1984), Neumann et al, Neuron 23:83-91 (1999)), Richardson et al, J. Neurocytol. 15:585-594 (1986)). The resection produced a peripheral nerve injury that affected DRG neurons on the same side as the lesion, but left the contralateral ganglia uninjured except for the spinal cord lesion itself (FIG. 5).

After 1-4 months, the fluorescent axonal tracer diI was introduced into the distal end of the nerve graft to label any neurons that had been able to regenerate their axons at least 5 mm into the graft. As expected from previous studies (Richardson et al, J. Neurocytol. 13:165-182 (1984), Neumann et al, Neuron 23:83-91 (1999)), dorsal root ganglia subjected to the peripheral nerve injury contained numerous labeled neurons (63±22 labeled neurons per ganglion, FIG. 5). For those ganglia, no difference was found between control (wild-type) and transgenic animals. This is not surprising, because the peripheral nerve injury induces GAP-43 and CAP-23, along with other growth-associated proteins, in the DRG neurons of both wild-type and transgenic animals.

The retrograde labeling procedure does not account for axons that may be competent to regenerate, but fail to encounter a direct tissue bridge between the spinal cord and graft tissue, are blocked from entering the graft by inhibitory molecules at the lesion site (Davies et al, Nature 390:680-683 (1997)), or grow around the lesion site rather than entering the graft (Neumann et al, Neuron 23:83-91 (1999)). To estimate the efficiency of the grafting procedure in identifying axons competent for regeneration, diI was applied directly to the spinal cord lesion sites to label all axons transected by the lesions. This direct spinal application labeled 372±60 neurons per ganglion. Thus, when DRG neurons are expressing the full complement of genes induced by peripheral nerve injury, approximately 17% (63/372) of spinal DRG axons successfully enter the graft and regenerate for at least 5 mm.

In the absence of a peripheral nerve lesion, however, adult DRG neurons of control (non-transgenic) mice were unable to extend their spinal axons into the nerve grafts. Ganglia on the side contralateral to the peripheral nerve lesion contained a mean of 0.4 labeled neurons per ganglion (n=5 animals), consistent with previous observations in rats (Richardson et al, J. Neurocytol. 13:165-182 (1984). Expression of GAP-43 and CAP-23 induced dramatic increase in the number of neurons that regenerated their spinal axons. In animals expressing both transgenes, 25±8 cells per ganglion were labeled in the absence of a peripheral nerve injury, more than 60 times as many as in controls (n=6 animals; p<0.0001). This means that approximately 7% of transected axons in the dorsal column were able to regenerate into and through the nerve graft.

As predicted by the in vitro assays, neither GAP-43 nor CAP-23 alone could elicit the regeneration triggered by the two transgenes together. Introduction of the peripheral nerve grafts into the dorsal columns of transgenic mice expressing either gene alone (n=2 animals each), resulted in retrograde labeling of only 1-2 cells per ganglion in the absence of peripheral injury. This labeling is not statistically distinguishable from non-transgenic animals in the absence of peripheral injury, but is dramatically less than in animals expressing both transgenes (p<0.0005). Retrograde labeling on the side subjected to peripheral nerve injury (55-88 cells per ganglion) confirmed that the grafting and labeling procedures were effective in these animals. Thus, the dramatic increase in spinal axon regeneration triggered by co-expression of GAP-43 and CAP-23 was not supported by either gene acting alone.

In animals expressing both GAP-43 and CAP-23, immunohistochemistry showed that almost all retrogradely labeled neurons express both of the transgenes (FIG. 4). Quantitation of the results from one ganglion showed that all retrogradely labeled neurons stained intensely for the chick CAP-23 protein, while 25 out of 26 diI-filled cells showed clear cell body staining for GAP-43 (FIG. 4). The remaining cell was surrounded by intense membrane-like staining for GAP-43, but intense staining of axons (FIG. 4) made it difficult to determine whether the transgene was expressed in the diI labeled cell or in neighboring neurons. Despite this ambiguity, the results show that the increase in spinal axon regeneration in animals expressing both GAP-43 and CAP-23 arises from individual neurons that express both transgenes within the same cell.

All documents cited above are hereby incorporated in their entirety by reference. Specifically incorporated by reference is Bomze et al, Nature Neuroscience 4(1):38-43 (2001).

What is claimed is:

1. A method of stimulating axon repair or regeneration at a central nervous system injury site in a patient comprising introducing into neuron cell bodies present at said injury site two or more members of a family of growth cone proteins that are missing or deficient in adult neurons, wherein said introduction is effected under conditions such that said stimulation is effected.

2. The method of claim 1 wherein said members have related but complementary functions in axonal growth cones.

3. The method of claim 1 wherein said members comprise GAP-43 and CAP-23, or analogs or functional portions thereof.

4. The method of claim 1 wherein at least one of said members is selected from the group of proteins consisting of MARCKS, MacMARCKS, paralemmin, GAP-43, CAP-23, and analogs and functional portions thereof.

5. The method according to claim 1 wherein at least one DNA sequence encoding said members is introduced into said neuron cell bodies under conditions such that said DNA sequence is expressed and said members are thereby produced.

6. The method according to claim 5 wherein said at least one DNA sequence is present in a vector operably linked to a promoter.

7. The method according to claim 6 wherein said vector is a viral or plasmid vector.

8. The method according to claim 7 wherein said vector is a viral vector.

9. The method according to claim 8 wherein said virus is a neurotropic virus.

10. The method according to claim 9 wherein said virus is a herpes, sindbis, polio, pseudorabies or adenovirus.

11. The method according to claim 5 wherein said at least one DNA sequence is introduced by lipofection.

12. The method according to claim 11 wherein liposomes used in said lipofection comprise cationic lipids chemically coupled to a targeting molecule.

13. The method according to claim 12 wherein said targeting molecule is a hormone, neurotransmitter or antibody.

14. The method according to claim 5 wherein said at least one DNA sequence is introduced as a naked DNA plasmid.

15. The method according to claim 1 wherein said members are introduced directly into neuron cell bodies present at said injury site.

16. A method of stimulating axon repair or regeneration at a central nervous system injury site in a patient comprising contacting neurons present at said injury site with an agent that activates expression of two or more members of a family of growth cone proteins that are missing or deficient in adult neurons, under conditions such that said stimulation is effected.

17. The method according to claim 16 therein said members comprise GAP-43 and CAP-23.

18. A method of screening a test compound for its ability to stimulate axon repair or regeneration comprising contacting adult neurons with said test compound and assaying for activation of expression of two or more members of a family of growth cone proteins that are missing or deficient in said adult neurons in the absence of said test compound, wherein a test compound that activates said expression is a candidate agent for use in stimulating axon repair or regeneration.

19. The method according to claim 18 wherein said members comprise GAP-43 and CAP-23.

20. A method of treating nerve damage associated with a lesion or a disease or dysfunction of the nervous system comprising administering to a patient in need of such treatment an amount of a compound identifiable by the method according to claim 18 as being able to stimulate axon repair or regeneration so that said treatment is effected.

21. The method according to claim 20 wherein said nerve damage results from a spinal cord injury, head trauma or stroke.

22. The method according to claim 20 wherein said nerve damage results from a neurodegenerative disease.
